(19) United States
(12) Patent Application Publication
(10) Pub. No.: US 2005/0033608 A1
(43) Pub. Date: Feb. 10, 2005

(54) Homoeopathic analysis and synthesis software

(76) Inventor: Jeremy Sherr, Malvern (GB)

Correspondence Address: G.E. EHRLICH (1995) LTD.;c/o ANTHONY CASTORINA, SUITE 207, 2001 JEFFERSON DAVIS HIGHWAY, ARLINGTON, VA, 22202, US

(21) Appl. No.: 10/815,768

(22) Filed: Apr. 2, 2004

Related U.S. Application Data
(60) Provisional application No. 60/491,993, filed on Aug. 4, 2003.

(51) Int. Cl. G06F 17/60; A61B 5/00

(52) U.S. Cl. 705/2; 600/300

(57) ABSTRACT

A system and a method for assisting homoeopathic practitioners in case taking and analyzing the symptoms to facilitate the perception of themes and patterns, and to guide practitioners towards selection of the most correct rubric(s), rubric combinations and remedies. Preferably, the present invention is capable of presenting a plurality of potential rubrics to the practitioner, more preferably with differential weighting according to the likelihood of a potential rubric or particular rubrics to be correct. Optionally and most preferably, the present invention is capable of assisting and guiding the homoeopathic practitioner throughout the entire homoeopathic process, including case taking, analyzing and synthesizing information, selecting the most preferred or correct group of rubrics, selecting a remedy and potency which is most appropriate to the case synthesis, performing follow-up and long term case management.

This application claims priority from U.S. Provisional Application No. 60/491,993, filed on 4 Aug. 2003, which is hereby incorporated by reference as if fully set forth herein.

### FIELD OF THE INVENTION

The present invention relates to a system and a method for analysis and synthesis of the symptoms of a patient with regard to the homoeopathic treatment system, and in particular, for such a system and method that is capable of assisting the physician to properly assess these symptoms, as well as of guiding and facilitating the entire homoeopathic process, optionally from case taking through to long term case management.

### BACKGROUND OF THE INVENTION

Homoeopathy, as it is practiced today, is a system of medical treatments that was developed by Samuel Hahnemann in 1790 (late 18^{TH} century). Dr. Hahnemann was dissatisfied with the paucity of successful cures in medical treatment and the toxicity of the available drugs. As an experiment, he took daily ingestions of cinchona bark (quinine), which was a popular medicine for malaria treatment and found that he developed the well-known symptoms of malaria. With this he recognized the principle of “like cures like,” known earlier to the medieval physician Paracelsus and to the ancient Egyptians and Greeks. This first and fundamental principle of homoeopathy (homeo—similar; pathy—suffering) states that substances that can cause certain symptoms in a healthy person can also cure similar symptoms in the sick.

Thus one of the cornerstones of homoeopathy is the Doctrine of Similars, meaning that drugs or other substances which cause particular symptoms in large amounts should be curative for these symptoms when taken in small amounts. Homoeopathy also relies upon remedies which have been tested on healthy individuals in order to determine their effects, which can then be used to treat sick individuals. This research was performed by giving the remedies to healthy individuals, and then recording the symptoms that each drug produced. The substances tested are taken from minerals, gases, plants, animals, diseased organs and medicines. This testing and recording of a homoeopathic drug on healthy people is called a proving. Provings are recorded and collated, and then assembled together in a reference text called a materia medica.

The text in which Dr. Hahnemann summarized his findings and philosophy is called the Organon of Medicine, a portion of which states (6^{TH} edition /part 3):

“If the physician clearly perceives what is to be cured in diseases, that is to say, in every individual case of disease (knowledge of disease, indication), if he clearly perceives what is curative in medicines, that is to say, in each individual medicine (knowledge of medicinal powers), and if he knows how to adapt, according to clearly defined principles, what is curative in medicines to what he has discovered to be undoubtedly morbid in the patient, so that the recovery must ensue—to adapt it, as well in respect to the suitability of the medicine most appropriate according to its mode of action to the case before him (choice of the remedy, the medicine indicated), as also in respect to the exact mode of preparation and quantity of it required (proper dose), and the proper period for repeating the dose: if, finally, he knows the obstacles to recovery in each case and is aware how to remove them, so that the restoration may be permanent: then he understands how to treat judiciously and rationally, and he is a true practitioner of the healing art.”

Many materia medicas have been developed according to this philosophy. The current materia medicas have over 4,000 proven drugs listed. The drugs are derived from minerals, plants, and animal substances. The remedies are listed in alphabetical order in the materia medica. The materia medica includes not just symptoms that were proven but also toxicological symptoms as well as symptoms that were actually cured in sick patients using that particular remedy. The symptoms in the materia medica are categorized in order from the top of the body down, such that the symptoms in the head are categorized together, then the eyes, ears, nose and so on until the extremities.

Some remedies have only very few symptoms listed in the materia medica, while others have 15,000 symptoms. Since it is impossible to remember all the symptoms of each remedy, about 150 years ago the information was collated into a reference form. All the remedies that affect a certain part of the body in a certain way were placed under a particular category. The book that contained these categories is called a repertory. The categories listed in the repertory are called rubrics.

The repertory of the materia medica is actually a reference tool that lists all the symptoms produced or cured, a particular category or rubric. For example, a rubric might list: Eyes; pain; general; right; motion aggravation, with one remedy listed under the rubric. Rubrics can be very specific like this example, or very general. A general rubric would be Eye; pain, and that general rubric would contain many hundreds of remedies.

Other important principles of homoeopathy include the principle of Totality of Symptoms. The principle of the totality of symptoms specifies that the whole array of symptoms be to be treated as one entity. As a holistic medicine, homoeopathy considers all the symptoms of a person as expressions of the inner disharmony or disease. No one symptom or group of symptoms can to be singled out and treated separately from the larger set of symptoms.

Another essential principle of homoeopathy is individualization, which means that each person is treated as an individual with unique symptoms. Rather than giving one medicine to every person suffering from a particular disease such as pneumonia, each person is studied for his idiosyncratic symptoms. For instance, one person may have a fever in the morning, one in the evening; one person's cough is better from cold drinks, another's cough is better from hot drinks. One person has also has pains in the knees, while another has trembling. Each person has a distinctive set of manifestations, which demands its own specially chosen medicine. The more precisely the medicine fits that unique individual disease, the more quickly, rapidly and safely that person will get better.

Yet another important principle is that of Minimum Dose. Homoeopathic medicines are administered in ultra diluted doses to assure gentle healing and to prevent unwanted effects. Through serial dilution and succusion called potentization, only the enhanced healing qualities of the substance remain after the poisonous or harmful chemical components of a substance are eliminated. When a patient takes a potentised homoeopathic medicine, called a remedy, there is much less danger of harmful symptoms or side effects developing as is likely with conventional drugs and medications.

The process of treating with homoeopathy may be broken down into the following steps:

The Homoeopathic Interview

Pre Analysis Evaluation

Case Analysis and Synthesis

Rubric Selection and Repertorisation

Materia Medica Study

Potency Selection and Dosage

Remedy Reaction and Follow up Evaluation

The next prescription

Long-term case management

Interspersed in this process is the treatment of acute and epidemic diseases.

Case Taking and Writing

The homoeopath conducts a lengthy and in-depth interview with the patient in order to gather all of the unique symptoms of the individual. Consideration is given to all levels, physical, mental and emotional, as well as past illnesses, traumas, personality and family history. Since individualization of symptoms is a key factor in homoeopathy, much detail and precision about sensation, location and modalities of symptoms are needed. If the patient suffers from headache, then the homoeopath needs to know specifically what the pain feels like, where the head hurts and what factors, such as position, light, noise, eating or motion, when the headache started, what make the pain better and what makes it worse. Furthermore the homoeopath will want to elicit many general symptoms such as appetite, food desires and aversions, weather preferences, sleep, dreams, perspiration, gynecological and sexual problems and more.

Every symptom is written down by the homoeopath using the patient's words.

Pre-Analysis Evaluation

Before analyzing the case, the homoeopath does a preanalysis assessment of the information that the patient has given. This includes evaluating the patient's overall health and vital energy, the presence of pathology and obstacles to cure, the organ affinities in the case, the acute or chronic nature of the disease, the influential miasm (chronic disease) and the prognosis both with and without homoeopathic treatment.

Case Analysis and Synthesis

The homoeopath must prescribe on the totality of the patient's symptoms, choosing those that represent the essence of the internal disease. In order to do this the prescriber must sort and analyse the collected symptoms to reach an understanding of the patterns that represent the inner disharmony in the patient. Representative patient symptoms will be matched to the corresponding symptoms of a proven homoeopathic remedy, taking into account any strange, rare and peculiar symptoms.

There are many approaches to case analysis and no one method will work for all cases. A case may require one or more methods of analysis in order for the homoeopath to understand “what needs to be cured.” Some analysis methods are: opposites, the circle of elements, analogy, Chinese physiology, chakras, and grammatical parameters (“the verb”, sensation and function).

After breaking the symptoms down and sorting them into categories, it is necessary to identify the common denominator running through all symptoms and analysis so as to synthesize the essential nature of the case (“that which is to be cured”)

Rubric Selection and Repertorisation

Once the homoeopath understands the case, he/she then decides which symptoms are most significant and distinguishing and translates these symptoms into rubrics.

Rubrics are codified symptoms contained in a reference book called a repertory. Each rubric lists all the remedies capable of causing or curing that particular symptom. With nearly 5000 remedies containing thousands of proving symptoms, most modern repertories contain over 800,000 symptoms Categories are arranged according to the organ systems of the body. For example, the rubric, “Ears; PAIN; pulsating, throbbing” lists twenty two remedies.

After all appropriate symptoms and their corresponding remedies have been selected, a mathematical sort is done by hand or by computer and remedies which are common to all or most of the symptoms are recommended.

If incorrect rubrics are entered, then incorrect remedies will be proposed. If many rubrics are entered, then many remedies with a large sphere of action are prominently weighted, while the more appropriate smaller remedies may be overlooked. Therefore it is most useful to enter a smaller number of precisely selected rubrics one at a time in order to obtain a more mathematically representative listing of remedies.

Materia Medica Study and Differential Diagnosis

The recommended remedies are then studied in the materia medicas (reference books listing all remedies and their symptoms), paying attention to the general description of organ affinities, modalities, physical, mental and emotional symptoms and causations. The remedy that is most suitable and fits the dynamics and the totality of the case is the one chosen for the prescription.

Potency Selection and Dosage

The potency of the prescribed remedy is based on several factors including strength and vitality of the patient, pathology and obstacles to cure. There is a wide range of potencies, which should be used, as appropriate. There are also a variety of approaches to the number of repetitions, ranging from a single dose to frequently repeated doses. Choosing the potency and repetition are part of the science and art of prescribing.

Follow-Up Evaluation and Remedy Reaction

The homoeopath conducts an in depth follow-up evaluation after an appropriate interval following the first remedy. Notation is made of remedy reactions, including any improvement, aggravation and change in symptoms. All changes in symptoms are compared to the original descriptions and questions are asked about any new symptoms or original symptoms that have not yet been mentioned in the follow-up.

There are three broad categories of reactions to a remedy: amelioration of symptoms, worsening of symptoms and no reaction. The ideal reaction is one of a slight intensification of existing symptoms or aggravation, followed by general amelioration. At some time the reduced symptoms will reappear and their return should be noted for the character, intensity and order of appearance. The patient should be dosed with the same remedy and potency as long as an improvement occurs. If there are no symptoms indicating another remedy, then the original remedy should be continued in ascending potencies and returning to lower potencies if needed. When symptoms change, then another remedy is chosen to match them. The more suitable the remedy, the closer to the ideal is the reaction.

When symptoms have worsened, then the chosen remedy was either a superficial remedy causing new and more serious symptoms to arise or else was the wrong remedy allowing the underlying disease process to continue unabated. In both of these situations a new remedy must be found going back to the original case notes and following the above process and steps. Likewise, when the patient feels no reaction, the case must be restudied and a new remedy must be sought. There are many shades of gray between these extremes. Interpreting the remedy reaction and choosing a subsequent remedy are fine and complex aspects of the science and art of homoeopathy.

Long Term Case Management

The patient continues in homoeopathic treatment as long as symptoms are present and treatment may range from a few months to several years. At each follow-up, symptoms are evaluated and the patient's progress and overall health are continually assessed. Remedies will be repeated as necessary and new remedies chosen when clearly indicated. It is expected that the patient will move through the accumulated layers of his chronic disease and each person's progression to health will be unique. This progress will be viewed by the practitioner through the understanding of miasms, or inherited patterns of disease. Patients may be treated from prenatal to old age, as the goal of homoeopathy is to stimulate the natural healing abilities towards a smooth, painless and meaningful progress throughout life.

As can be seen from the above description, essentially, homoeopathy is a very simple medicine to practice, as the physician simply tries to perfectly match the symptoms of the patient to a remedy. There are, however, a few difficulties. For instance, it may be impossible to easily match all the symptoms of a patient to a remedy. If all symptoms are given equal weight, the remedies with many rubrics are more likely to be selected than those with fewer rubrics, such that the repertory has a natural tendency to show the large remedies as the right remedies. In addition, rubrics chosen may be unrelated or superficial, they may not represent the true nature of the disease, and they may not be grouped in a proper manner. Choosing the remedy from the resulting list of remedies is also a complicated and subtle procedure. It is not always easy to know which aspects of the case should match which aspects of the remedy, as there are countless possibilities and combinations. Differential diagnosis of similar remedies is often very subtle, and interpreting remedy reactions may be complex. These factors make homoeopathic success a challenging and difficult task. It is essential to master every step of the process in order to achieve the wonderful results homoeopathy is capable of bestowing.

Therefore, the practitioner must be able to understand the essential nature of the case's totality and to logically consider which symptoms truly represent the whole disease state of the patient. This means that the practitioner must be able to sort through these symptoms and consider which symptoms are the most important and how to group them. Thus, the ability of the practitioner to correctly sort through the symptoms also depends upon the practitioner being able to correctly assess all of the symptoms of the patient.

Because homoeopathy requires all of the symptoms of the patient to be assessed, unlike Western medicine in which specific symptoms are addressed and then treated, the case-taking skills of the practitioner are extremely important. Failing to correctly note and place particular symptoms may cause the homoeopathic practitioner to completely misdiagnose the patient. Such a misdiagnosis in turn causes the incorrect remedy to be given to the patient. There can only be a small number of remedies that will benefit a patient at one time.

For example, the anamnesis or case taking must be performed well, in order for repertorization (translation of the words that the patient spoke into symptoms/rubrics listed in the Repertory and determining which remedies were listed in the greatest number of rubrics listed in the repertory) to also be performed well. If repertorization is not performed well, then the final selection of the remedy may fail to cure the patient. Once repertorization suggests a group of remedies, careful selection and differential diagnosis must occur. Thus, it is essential to have a profound grasp of the entire case and all its themes and patterns, and to be able to match these to a remedy. Unlike Western medicine, in which the correlation between the symptom to be treated and the drug to be selected is fairly clear, homoeopathy requires a greater analysis of the symptoms of the patient.

Previous attempts to design computer software to aid homoeopathic practitioners have not been successful, at least in part because they focused too narrowly on crossing symptoms and rubrics to find remedies, rather than focusing on the homoeopathic process itself. For example, repertories were first computerized into several databases to speed up the search process a number of years ago. Software has also been created which attempts to help the practitioner select the proper remedy. For example, U.S. Pat. No. 5,797,839 to Herscu provides a method for selecting the proper remedy according to the symptoms entered to the software. Herscu also isolates another minor aspect of the homoeopathic process according to his own analysis methodology, which is recognizing the patient's pathology as a cycle of phases that repeats it self in different forms. Some ability to group symptoms is also provided. However, the method of Herscu does not provide support and guidance for all, or most aspects of the homoeopathic process.

Indeed, all of these attempts at computerization suffer from a number of drawbacks due to their extremely narrow focus. Since homoeopathy differs from Western medicine in that the process of homoeopathic case taking, analysis and diagnosis (synthesis) is at least important, if not more important so as to reach a “goal” with regard to the selection of a remedy and correct long term case management, this narrowly focused approach may actually result in the selection of a less suitable or even non-suitable remedy. Furthermore the delicate and complex processes of actually choosing rubrics, of potency selection, case follow ups, subsequent prescriptions and long term case management are not addressed by existing software.

### SUMMARY OF THE INVENTION

The background art does not teach or suggest a solution to the problem of organizing and analyzing free flowing information, particularly when such information is obtained from another human being as part of a professional work. Examples of professional work in which such free flowing information is obtained include, but are not limited to, medical practice, legal practice, professional consulting such as for architecture, psychological counseling, interviews, scientific exploration and education.

One example of a field in which the background art fails is for assisting homoeopathic practitioners to organize and analyze large amounts of information obtained during case taking from a patient. Unlike Western medicine, which focuses upon diseases and therefore may be practiced according to a more defined set of extracted information, although this may not be optimal, homoeopathic practice requires the practitioner to examine the patient as a whole, and therefore to treat the patient as a whole.

The background art does not provide a suitable solution which can guide and assist in analyzing and synthesizing the information obtained from the patient, assist in pattern and theme recognition, assess and refine repertorization, assist in performing differential diagnosis, choosing potency, assess follow-up information, choose a second or further prescriptions, and treat acute cases and epidemics in relation to the chronic treatment.

All existing software for assisting the homoeopathic practitioner focuses on the crossing of rubrics to obtain lists of possible remedies, or searching through the materia medica (remedy database). This represents only a fraction of the entire homoeopathic process and practice.

The present invention overcomes these problems of the background art by providing a system and a method for organizing the free flow of information as given through a communication with a human being for the purpose of consultation by at least one other human being. Hereinafter, the term “consultation” includes one or more activities performed as part of a professional work for a project, termed herein a “case”. Examples of professional work in which such free flowing information is obtained include, but are not limited to, medical practice (in which the case is a patient), legal practice (in which the case could be a legal or court case, a patent application, a trademark application, a legal opinion and so forth), professional consulting such as for architecture (in which the case could be a building to be built or modified), psychological counseling and education. The present invention is operative for organizing the free flow of information obtained through the communication with a human being for any of these different types of consultative services, and/or any other services having these characteristics. By “organizing”, it is meant that the present invention is capable of processing the information according to a plurality of different levels of processes, starting from a more general level and moving to at least one more specific level. Each process preferably includes some type of analysis, depending upon the nature of the information and of the consultation. More preferably, a highly specific level of process is also performed, in which all or at least a significant portion of the details of a case are organized and preferably displayed to the user. Also more preferably, the general level requires the user to summarize the essence of the collected free flow of information in a targeted manner, according to the goals and/or characteristics of the consultation.

Next, the present invention preferably at least assists in the selection of a solution, and may also automatically select the solution, according to the analyzed information. The present invention then preferably compares the solution(s) to the analyzed information and also preferably to the raw information, for determining suitability of the solution(s). More preferably, the present invention is capable of detecting a non-suitable and/or less suitable solution, and of alerting the user to the problematic solution(s).

Optionally, the present invention may also assist with follow-up and long term management of a case.

According to one embodiment of the present invention, the method and system are capable of coaching and assisting a homoeopathic practitioner for at least an initial portion of the homoeopathic process, but preferably throughout the homoeopathic process, as a homoeopathic expert system. Preferably, the present invention at least features a case taking mechanism, which preferably assists the homoeopathic practitioner to organize the free flowing information into a plurality of categories. Optionally, a plurality of organizational processes are supported, such that the free flowing information is preferably first categorized according to general categories, and then according to more specific categories. At each stage, preferably the organized information and categories are represented graphically for facilitating understanding and manipulation of the information and categories. The organizational process more preferably culminates in a synthesis of information, optionally and more preferably to determine a plurality of synthesized concepts.

The present invention may optionally only be implemented and/or operated according to the organizational process(es). More preferably, however, the present invention further provides matching of the synthesized concepts to a remedy database. Affinities obtained during the synthesis of the information are also preferably matched to the remedy database. The rubric(s) obtained during this matching process are preferably displayed, more preferably according to weights, for example for determining relevance, likelihood or any other relationship between the rubrics and the previously organized information. Optionally and more preferably, the rubrics themselves are organized, optionally automatically but at least preferably semi-automatically. Statistical evaluation of the process and expert advice will be provided as a feedback mechanism, as described in greater detail below.

Next, possible remedies are preferably provided to the practitioner according to the organized rubrics, preferably automatically or at least semi-automatically. More preferably, these possible remedies are analyzed with regard to the analyzed information and synthesized concepts, and also optionally with regard to the initial comparison to the database of possible remedies.

Optionally and more preferably, the process further includes assistance with the choice of potency.

According to other preferred embodiments of the present invention, follow-up visits by the patient to the practitioner and long term case management are also provided. Other optional embodiments include but are not limited to, second and subsequent prescriptions, treatment and management of patients with acute disease, chronic diseases and also epidemics. Furthermore the present invention will optionally and preferably supply statistical and graphical feedback and advice pertaining to the entire homoeopathic process. The present invention will also be capable optionally and preferably of using its case taking software for the analysis and synthesis of new provings, and for gathering clinical information on materia medica gained through the homoeopathic process.

Preferably, the present invention is capable of presenting the patient's data in logical clusters and formats which enhance pattern formation and recognition, and which therefore may lead to more accurate rubric and remedy selection, optionally as well as long term case management. Optionally and most preferably, the present invention is capable of prompting the practitioner to query the patient in a more efficient manner, and may also optionally provide an artificial intelligence system that can optionally aid and/or tutor the homoeopath throughout the homoeopathic process.

Optionally, the present invention may also suggest a remedy, but this function may also not be implemented and/or may only be partially implemented, because the preferred emphasis of the present invention is upon the correct analysis of symptoms to produce rubrics.

### BRIEF DESCRIPTION OF THE DRAWINGS

The invention is herein described, by way of example only, with reference to the accompanying drawings, wherein:

FIG. 1 is a block diagram of an exemplary system according to the present invention;

FIG. 2 shows an exemplary method according to the present invention;

FIG. 3 shows an exemplary window in a GUI according to the present invention, in which the practitioner is able to enter the symptoms of the case;

FIG. 4 shows an exemplary window in a GUI according to the present invention, in which the information from the GUI in FIG. 3 is automatically and/or semi-automatically distributed to the appropriate boxes (windows) according to homoeopathic context;

FIG. 5 shows an exemplary window in a GUI according to the present invention, in which the practitioner is able to select concepts and affinities, and to import concepts, affinities and symptoms from the case taker module for the selection of appropriate rubrics; and

FIG. 6 shows an exemplary GUI for entering or selecting symptoms according to the present invention.

### DESCRIPTION OF THE PREFERRED EMBODIMENTS

The present invention is of a system and a method for organizing the free flow of information as given through a communication with a human being for the purpose of consultation by at least one other human being. Hereinafter, the term “consultation” includes one or more activities performed as part of a professional work for a project, termed herein a “case”. Examples of professional work in which such free flowing information is obtained include, but are not limited to, medical practice (in which the case is a patient), toxicological reports, legal practice (in which the case could be a legal or court case, a patent application, a trademark application, a legal opinion and so forth), professional consulting such as for architecture (in which the case could be a building to be built or modified), psychological counseling, interviews and education. The present invention is operative for organizing the free flow of information obtained through the communication with a human being for any of these different types of consultative services, and/or any other services having these characteristics. By “organizing”, it is meant that the present invention is capable of processing the information according to a plurality of different levels of processes, starting from a more general level and moving to at least one more specific level. Each process preferably includes some type of analysis, depending upon the nature of the information and of the consultation. More preferably, a highly specific level of process is also performed, in which all or at least a significant portion of the details of a case are organized and preferably displayed to the user. Also more preferably, the general level requires the user to summarize the essence of the collected free flow of information in a targeted manner, according to the goals and/or characteristics of the consultation.

Next, the present invention preferably at least assists in the selection of a solution, and may also automatically select the solution, according to the analyzed information. The present invention then preferably compares the solution(s) to the analyzed information and also preferably to the raw information, for determining suitability of the solution(s). More preferably, the present invention is capable of detecting a non-suitable and/or less suitable solution, and of alerting the user to the problematic solution(s).

Optionally, the present invention may also assist with follow-up and long term management of a case.

According to one embodiment of the present invention, the method and system are capable of coaching and assisting a homoeopathic practitioner for at least an initial portion of the homoeopathic process, but preferably throughout the homoeopathic process, as a homoeopathic expert system. Preferably, the present invention at least features a case taking mechanism, which preferably assists the homoeopathic practitioner to organize the free flowing information into a plurality of categories. Optionally, a plurality of organizational processes are supported, such that the free flowing information is preferably first categorized according to general categories, and then according to more specific categories. At each stage, preferably the organized information and categories are represented graphically for facilitating understanding and manipulation of the information and categories. The organizational process more preferably culminates in a synthesis of information, optionally and more preferably to determine a plurality of synthesized concepts.

The present invention may optionally only be implemented and/or operated according to the organizational process(es). More preferably, however, the present invention further provides matching of the synthesized concepts to a remedy database. Affinities and concepts obtained during the synthesis of the information are also preferably matched to the remedy database. The rubric(s) obtained during this matching process are preferably displayed, more preferably according to weights, for example for determining relevance, likelihood or any other relationship between the rubrics and the previously organized information. Optionally and more preferably, the rubrics themselves are organized, optionally automatically but at least preferably semi-automatically. Optionally and more preferably statistical feedback and expert advice are provided for the selection of rubric groups.

Next, possible remedies are preferably provided to the practitioner according to the organized rubrics, preferably automatically or at least semi-automatically. More preferably, these possible remedies are analyzed with regard to the analyzed information and synthesized concepts, and also optionally with regard to the initial comparison to the database of possible remedies.

Optionally and more preferably, the process further includes assistance with the choice of potency.

According to other preferred embodiments of the present invention, follow-up visits by the patient to the practitioner and long term case management are also provided. Other optional embodiments include but are not limited to, treatment and management of patients with chronic diseases and also epidemics, as well as management of acute disease (epidemic or otherwise). Also preferably and optionally, the software will be used for the recording, analyzing and synthesizing of provings.

Preferably, the present invention is capable of presenting the patient's data in logical clusters and formats which enhance pattern formation and recognition, and which therefore may lead to more accurate rubric and remedy selection, optionally as well as long term case management. Optionally and most preferably, the present invention is capable of prompting the practitioner to query the patient in a more efficient manner, and may also optionally provide an artificial intelligence system that can optionally aid and/or tutor the homoeopath throughout the homoeopathic process.

According to preferred embodiments of the present invention, the system of the present invention preferably features a number of modules for implementing the analysis of the symptoms of the patient. Optionally and preferably, a first module is the case taker. This module is also preferably used for performing the first stage of the method of the present invention, for case taking.

As the practitioner records and types in the patient's medical history, the information is preferably automatically, semi-automatically or manually sorted into homoeopathically relevant categories. The sifted and sorted information can optionally then be presented in a variety of views (windows) so as to enhance analysis of the data and its use in solving the case and also preferably for case management. For example, all pathological information may optionally be stored in one ‘window’. Related words and idioms are automatically grouped and stored in one window. All relevant life events may be displayed on a time line, since as described in greater detail below, the relative time at which particular symptoms occur is important. Logical connection and analogies are automatically detected or tagged, then grouped and displayed for easy reference. Diet and other lifestyle issues are sorted into their relevant windows. Other homoeopathically relevant information such as weather and temperature influences, times of day, appetite, sleep, emotional and intellectual symptoms and many others are sorted into categories. Body sections are sorted, such that all bladder symptoms preferably appear together. There are many other parameters by which data will be sorted, partly automatically and partly with some practitioner input. Another category preferably includes analyzing the statements of the patient grammatically, for example by noun, verb, adverb and so forth, and/or other methods of language analysis, as well as a word count and display and analysis of frequently appearing words and idioms.

According to optional preferred embodiments of the present invention, in order to assist the practitioner to enter the necessary information, preferably an electronic “tablet” and/or a PDA or any other type of handheld device or portable device, is provided as part of the system of the present invention, for enabling the practitioner to enter information with handwriting. Such tablets are well known the art, and may optionally be connected to a computer for example, in order for the handwritten notes of the practitioner to be automatically translated into data for analysis according to the present invention.

The entire case can then optionally be displayed in a variety of modes. Databases of pathology, diagnosis and other relative data will be readily available in a computerized format.

The case taker preferably includes a module for inputting, storing and searching the patient's personal details. This module also preferably automatically calculates the patient's age and creates a time line. It also automatically generates a variety of more esoteric information based on the date of birth (astrology, Chinese astrology, numerology, i-ching, tarot, etc). These features are optionally and preferably displayed as part of the analysis view as described below.

As described in greater detail below, stage 1 may optionally be split into two stages (stages 1 and 2): a first stage for registering the patient and obtaining personal details, and a second stage for taking the case.

Once the medical data has been sorted (preferably at least partially automatically), stage 3 of the method of the present invention preferably allows further manipulation of the data so as to form an even more comprehensive analysis of the case. Stage 2 may also optionally be performed by the case taker module of the system according to the present invention.

Stage 3 may also optionally and preferably be split into two stages as described in greater detail below, in which a first part would feature pre-analysis of the information for a general view of the information, more preferably including a synopsis of the essence of the information; and a second stage for analysis of the information (stages 3 and 4).

The analysis process may optionally include clustering the symptoms. More preferably, the symptoms are clustered into weighted groups, according to the relative importance and/or likelihood of each cluster. This aspect of the present invention is particularly preferred since symptoms may optionally be featured in more than one cluster, although the suitability of such clusters may differ. Preferably, this stage is performed by an analysis module according to the method of the present invention.

The various windows (clusters, groups,) can be manipulated so as to enhance analysis and synthesis of the case. This is preferably aided by a variety of graphics. Information appearing in the various windows is preferably tagged to their origin in the case and may be highlighted in the case by clicking on the link.

In stage 5, a synthesis process is preferably performed, in which the practitioner is prompted and aided in searching for a precise definition of the illness (Homoeopathic diagnosis or in homoeopathic terminology ‘what is to be cured’ (Organon of Medicine by Samuel Hahnemann 6^{th} edition).

It should be noted that stages 1-5 can also be utilized for the recording, storing and analyzing of homoeopathic provings, which are the records of symptoms experienced by healthy volunteers and collected toxicologies for the purpose of determining the remedy action. These provings are an integral part of the homoeopathic process. Thus the module can assist in the sorting, pattern recognition and synthesis of this very diverse information. In stage 6, in order to assist with the analysis, synthesis and remedy finding processes, optionally and preferably the system according to the present invention also features a new database of meta-concepts and physical affinities, optionally in addition to existing databases which feature individual symptoms. The concepts and affinities of the case will be automatically derived from the case and compared with these databases.

Homoeopathic affinity is a physical location or physiological system, eg, throat, ear, foot, digestion, metabolism, reproductive and so forth, as described in greater detail below. A homoeopathic concept is a generalized issue which characterizes the mental and emotional nature or pathology of the patient, i.e. religion, victim, forsaken, money, fear, as described in greater detail below.

In stages 7 and 8 a full list of possible rubrics is selected from the existing rubric database, and assistance is provided in selecting the most efficient rubrics. Stage 9 assists in grouping the most efficient composition of rubric groups.

In stages 8 and 9, rubrics are preferably selected and grouped according to the analysis provided during the synthesis stage, according to the clusters of the symptoms that are determined in that stage. As previously described, symptoms may potentially be associated with more than one rubric, yet not all such associations are likely to be correct. There is a science and art to the choosing a grouping of rubrics. This process is preferably performed by a rubric selection module which provides guidance and feedback on the choice of rubrics according to the system of the present invention.

The rules guiding this and other stages are preferably capable of updating and enhancing, for example through actions of the user, upgrades, and intelligent self upgrading, preferably through an analysis of the actions of the software and/or other implementation of the method of the present invention, as compared to the outcomes (preferably at each stage) selected by the practitioner, and/or by comparing success in treatment to the type of recommendations made.

Stage 10 preferably performs repertorisation, a process which is known in the art and for which software is available. Stage 11 may also optionally and preferably offer statistical and strategic analysis of the process, providing feedback and expert advice which can help the practitioner improve their choice of rubrics.

In stage 12, data from the case analysis module is optionally (but not necessarily) automatically compared with existing databases and repertorisations to arrive at possible remedies. Such a selection may also optionally be made manually by the practitioner. For example keywords form the case taker will be automatically exported to existing materia medica databases for wordsearches, and the result used to filter repertory results. Other filters may preferably and optionally include affinities, concepts and miasms.

In stage 13, preferably remedies resulting from the process are compared with the results of the analysis and synthesis stages of the case preferably from stages 4, 5, and 6.

Stage 14 preferably assists with differential diagnosis of possible remedies.

Stage 15 will prompt the practitioner for remedy choice justification to enhance clarity and for further reference.

Stage 16 will facilitate the choice of dose and potency.

In stage 17, follow-up visits to the practitioner by the patient are preferably examined in a specifically modified version of stage 2, the case taker. Original symptoms from stage 2 will be semi automatically or preferably automatically tagged to symptoms in stage 17, as well as to rubrics in stages 7,8,9 and 10. Indeed, such tagging may optionally and preferably be performed throughout the above process between stages. Tagging of symptoms between the various stages allows easy cross-reference and moving or “jumping” between symptoms. Preferably and optionally symptoms will be tagged and cross referenced between any two or more of stages 2, 4, 6, 7, 8, 9, 10, 11, 14, 16, 17, 18, 19, 20, 23, or 24. Symptoms will likewise optionally be tagged to materia medica stages such as in stages 14 and 28 for example.

In stage 18, once the patient returns, a comparison of the original case with results of the remedy is preferably performed, expressed by % of improvement or deterioration and optionally represented by graphs. This stage then preferably facilitates the stage of choosing a new remedy.

Stage 19 will assist and provide expert advice in the selection of the second and subsequent prescriptions, by utilizing information from stage 18, a variety of laws and the relationship of remedies database.

Stage 20 preferably involves a repeat of stages 17-19 for the purpose of all subsequent consultations.

In stages 21 and 22, the present invention also preferably comprises a module for case management, which more preferably allows comments and hints on long-term case management and tracks the progress of the patient in comparison to conclusions derived in stages 3-5 and stages 17-20. Stage 22 preferably specifically relates this information to progress through miasmatic layers of disease.

Stages 23 and 24 will enhance the treatment of acute and epidemic disease, by utilizing guidelines, rules and related repertories, clipboards and databases.

Stage 25 preferably supplies data and feedback the practitioner on his/her practice during the session and also for case management, and/or for any other aspects of the homoeopath's practice. The method at this stage preferably enables automatic monitoring of results, trends and patterns in terms of the patient's details such as age and professions, remedies, potencies, remedy results, number of rubric taken, remedy chosen to remedies suggested, potency chosen to potency suggested, number of consultations etc. Every stage of the module preferably provides feedback and is preferably and optionally displayed in numerical, percentage, verbal and graphical manner so as to facilitate a continual overall assessment of the practice.

Stage 26 preferably enables the practitioner to receive information about the recommendations of other practitioners for patients having the same or similar symptoms. For example, the present invention may optionally connect to a database, for example through a network such as the Internet. Optionally, the practitioner is only allowed to access the database if the practitioner also supplies information to the database, preferably coded to maintain the privacy of patients and the confidentiality of the practitioner/patient relationship. Therefore, in this stage the practitioner may optionally perform data mining for example, in order to learn more about how particular remedies are handled, for example in terms of dose and/or potency for example.

Stages 27-29 are not all necessarily performed, nor are they necessarily performed sequentially (with each or with other stages of the method as previously described); they represent different options for augmenting the method of the present invention.

Stage 27 is optionally performed to assist the practitioner if all attempts at selecting a treatment and/or previous treatments fail. In this stage, alternative pathways are suggested if previous suggestions fail. For example, this stage optionally and preferably features reviewing the selections and choices made by the practitioner, in order to suggest an addition question to ask, and/or a different way to analyze the information and/or a different pathway to follow to select a rubric and/or remedy. For example, the practitioner may optionally be provided with a flowchart of different possible pathways to consider and preferably their potential consequences in terms of treatment.

Stage 28 is optionally and preferably used to accumulate data regarding remedies gained from clinical experience and feedback during the entire homoeopathic process, and specifically stage 25. This information can then be added to existing databases and repertories, and optionally shared over a network such as the Internet with other users of the software.

Stage 29 preferably involves adding data from a new proving. As previously described, each proving preferably features gathering data from the effect of a substance on a plurality of healthy individuals, and then analyzing the effect(s) to determine the potential use(s) of the substance for treating patients. Stage 29 may optionally be supplied through an additional module, which would enable intake of all of the data on the effect(s) of the substance and analysis of the data. This module may optionally be a “stand-alone” module, as stage 29 is optionally performed separately, without any connection to the rest of the previously described method of the present invention. However, as this stage is based on stages 2-5, it can optionally and preferably add information directly into existing databases and repertories, specifically those mentioned in stages 6 and 10.

The present invention also preferably comprises a module for case management, which more preferably allows comments and hints on long-term case management.

According to preferred embodiments of the present invention, as previously described, since one of the most important aspects of case taking is determining the symptoms of the patient, the homoeopathic practitioner preferably considers a number of different factors, including but not limited to, the discernible body constitution of the patient; mental and emotional characteristics; occupation, lifestyle and habits, civic and domestic relationships (relationships outside and within the home), age, sexual function, etc. Other important factors to be considered preferably include but are not limited to, the timeline of the disease progression, the etiological constellation (the causations) and chronic miasms, and the seven constitutional factors. Other important factors to be considered preferably include but are not limited to, the affinities of the case, the timeline of the disease progression, the etiological constellation (the causations) and chronic miasms.

Another such issue/concept is mental and emotional character, civic and personal relationships, which includes the family situation as well as social relationships.

Other information preferably includes the objective signs, coincidental befallments and subjective symptoms are recorded in detail. The innate constitutional body types are to be examined during case taking, (especially in chronic diseases). This category includes Hippocratic constitution and a description of the physique (tall or thin, short or fat, etc) and the state of the vitality (weak, strong, unstable, etc). It also included the psoric, syphilitic and sycotic (as well as other miasmatic doctrines) constitutions.

The second such rubric is mental and emotional character.

The third such rubric is the occupation of the individual. The occupation that a person chooses is often characteristic of the individual's innate talents and desires. It also is an area which reveals many maintaining causes and occupational hazards, which may feed or maintain the disease state. These are general symptoms as they are characteristic of the whole.

The fourth such rubric is lifestyle and habits. These are cardinal general symptoms, and may include the likes and dislikes of the individual, such as food or weather preferences. They are clear general symptoms.

The fifth such rubric is civic and domestic relationships. This includes the family situation as well as social relationships. How a person relates to their mates, family, friends, and society in general, are very important source of general symptoms. To understand the full picture the homoeopathic practitioner must speak to the family, friends and colleagues of the patient. These are valuable general symptoms.

The sixth such rubric is age. Stage of life is a very important part of time and progression in homoeopathy. Some remedies work particularly well on babies while other are more suited to the elderly. Some work well at both extremes of life.

The seventh such rubric is sex and sexuality. Some remedies are more characteristic of females while some are more reflective of males. Some cover problems unique to the female and vice versa. Also the sexual nature, attitudes and health of an individual reveal information about the patient. Sexuality is an important part of the general symptoms as it is closely linked with the instinctive vital force and the emotional disposition.

The principles and operation of the system and method according to the present invention may be better understood with reference to the drawings and the accompanying description.

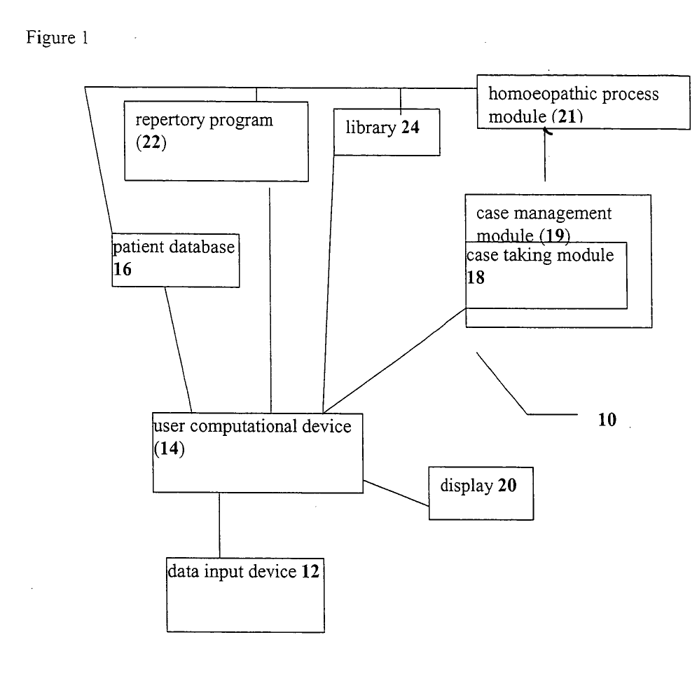

Referring now to the drawings, FIG. 1 shows an illustrative system 10, in which a user (such as a homoeopathic practitioner) interacts with a data input device 12 for entering information about a patient for case taking. As previously described, optionally and preferably data input device 12 comprises an electronic tablet for entering information in a handwritten form, the handwriting typically being performed with a stylus. Alternatively, data input device 12 may optionally comprise a tablet PC computer, a PDA or other handheld computing device, or a keyboard and/or mouse for a computer.

In any case, the information being entered through data input device 12 is preferably passed to a computational device 14 in communication with data input device 12. Computational device 14 may optionally be able to store the information, for example in a patient database 16. Preferably computational device 14 operates a case taking module 18 for performing intake of patient case information, and also preferably for displaying relevant information to the practitioner through a display 20 of computational device 14.

A case management module 19 preferably provides overall control of the processes. The actual processing is preferably performed by a homoeopathic process module 21, which preferably sorts the entered information into different “windows” or views, which are then preferably displayed to the practitioner through display 20. Optionally and preferably, the entered information may also be designated by accelerators or short cut keys to a second case form that is arranged with columns (CCF), which may also optionally be displayed to the practitioner through display 20. The accelerators designate the information to the right columns. Some of the accelerators serve in parallel for both windows and CCF.

System 10 also preferably features a repertory program 22 for arranging the symptoms entered through homoeopathic process module 21 into a plurality of groups or clusters. In this process, the information in the windows is preferably easily edited and shifted between the windows, thereby preferably permitting maximum flexibility while working with them.

Optionally and preferably, homoeopathic process module 21 may cause the information to be displayed to the practitioner according to different formats. For example, optionally information may be requested from the practitioner in the free case form (FCF), which is preferably similar to a word processing document. The user types in the information of the case, for example. Preferably, a keyboard accelerator (or typing code) is used to identify the type of information that follows in order to put it in the corresponding category (Ex: A for Affinities, C for Concepts). The accelerator (or keyboard shortcut) which may optionally comprise one or more letters for example or other characters and/or formatted characters, can preferably be typed anywhere within the sentence (at the beginning, in the middle or at the end). The result is preferably the same: the sentence is sent to the selected window(s).

Preferably, a keyboard accelerator (or typing code) is used to identify the type of information that follows in order to put it in the corresponding category (illustrative examples include but are not limited to CTRL-A for Affinities, CTRL-C for Concepts). This process of categorizing symptoms into clusters may also occur automatically, based on key words which are predefined, but can easily be added to by the user.

Also optionally, information being entered through the FCF may be marked with a color associated with a particular window, thereby causing the information to be sent to that window, or at least changing the color of highlighting of the displayed information. This supports the implementation of a “theme” function. For example, a new window “water” may be created and associated with the color blue. The patient says “I perspire a lot”, “very thirsty”, “tearful”. All those parts of text are preferably marked in blue and automatically copied to the “water” window, and vice versa.

The column case form (CCF) is preferably determined as a structured 1-4 column table (for example, for a 4 column table, the columns may optionally include section, sensation, modality and text). This represents the classical Kentian way of taking a case or writing a case. Kent was one of the leading homoeopaths of the last century, and explains this system in his book ‘Lectures On Homoeopathic Philosophy’. The information in the FCF is preferably channeled to the appropriate section of the CCF automatically or by using short cut keys.

| SECTION | SENSATION | MODALITY | TEXT |
|---|---|---|---|
| Head | Pain | Worse | in the night, the pain is very |
| | | | intense and I can't sleep |
| | | Nws (never | head injury |
| | | well since) | |

If the section name or accelerator are not typed in, the information preferably does not flow to column case form or any window; more preferably the information is marked with a function to highlight the undesignated information in the FCF. Any information not sent to CCF but sent to windows will be marked with one pound key # with a function to highlight all this type of undesignated information in the FCF. Therefore, if any of the information in the FCF is ‘missed’, the information is preferably marked to indicate this.

As previously described, the various types of information entered while taking the case are dispatched to one or more selected windows, which are also preferably displayed on display 20. Also, optionally and preferably, display 20 features a list of possible further question the practitioner may wish to ask, self generated during case taking, for listing information about further questions that the practitioner wishes to ask the patient at the end of the case taking process. An accelerator is preferably used for selecting the FAQ and entering information in it. A shortcut key causes a list of possible homoeopathic questions to be displayed. If any of these questions are selected the question will automatically be transferred to the FCF.

The information in the FAQ and/or the windows is preferably fully editable, for example through data input device 12. Optionally and preferably, information may be sorted by sections in the CCF. Also preferably, information that enters more than one window is marked, more preferably according to the number of windows entered.

Preferably, the practitioner can move information between windows by dragging with a mouse or other pointing device or key-board command, and may optionally also collect several items by command for moving at one time.

Also optionally and preferably, intensity may be indicated through different types of character formatting, such as underlining, bold and so forth. Such intensity is preferably enterable through one of the above case information formats, and is also preferably displayed to the user in the windows.

The user can also preferably add extra windows and to specify which accelerator is to be associated with this window. Each window preferably has an associated general list of questions (30-50). Pressing a dedicate key (for example the TAB key) preferably brings each window in the sequence to be displayed on display 20. Some of the windows may optionally be connected and may therefore receive input at the same time.

Each window is preferably associated with an abbreviation. The abbreviation is intended to identify the window the information should enter. The case form is preferably word sensitive to the name of each section (=chapter in repertory), part of the sentence or where appropriate the complete sentence till the next ENTER is copied to the corresponding section part in the CCF. The recognized word can be displayed in another color in the case form (with an option to deactivate).

The affinity window screen (preferably as shown on display 20) may then present the sections that received the most points. Each section represents a rubric, in the affinity repertory which is to be created and/or which may also be user definable.

Scoring is preferably performed by automatic and/or manual calculating of the frequency and intensity of each affinity. Each input symptom may count as one point unless it was intensified. Each one of the first sections presented in the Affinity window screen is preferably presented in descending order in terms of the score.

According to optional but preferred embodiments of the present invention, the user also preferably has the ability to view also all other sections of the affinity window on request through this screen display. The affinity window preferably has a direct link in to CCF relevant information; for example, if the user double clicks on the stomach section in the affinity window, the relevant or all stomach-related information in the CCF is preferably displayed.

Affinity window sections list may optionally include the following sections: Head (includes Headache and Vertigo, Face), Eye (Includes Vision), Ear (Includes hearing), Nose, Mouth, Teeth, Throat (Includes External Throat, Larynx), Stomach, Abdomen, Rectum, Urinary (Includes Bladder, Kidneys, Prostate, Urethra, Urine), Prostate, Genitalia (Male and Female), Respiration (Includes Cough), Expectoration, Chest, Back, Extremities, Perspiration, Skin, Mucous membranes, Discharges (includes Expectoration, Coryza, Leucorrhea, Cattarh, Mucous), Digestion (Stomach, Abdomen, Rectum, Stool), Heart, Circulation, Pelvic organs (Includes Anus, Rectum, Uterus), Fever includes Chill, Metabolism, Hormonal, Nerves, Joints, Muscles and empty windows (to create new types of affinities).

Other types of windows include information related to concepts, which are generalized mental or emotional characteristics or pathologies such as religious, money orientated, victim, aggressor, forsaken. A predefined list may optionally be used; preferably these are also user definable. These concepts are innovative as they contain generalized information rather then specific information, such that rather than the conventional designations of avarice, delusions of being poor, extravagant, dreams of money etc, there will be a meta concept rubric covering all money issues.

A further innovation regarding concepts and affinities is that each of these meta-concepts and affinities will be represented by two rubrics. The first and smaller primary rubric will contain the most essential remedies in which the subject i.e. money, digestion) is a major feature, while the second larger rubric will contain any remedy that may feature that subject even to the slightest degree. By entering these concepts and affinities, the user, by means of a sliding scale, can find an average between these two rubric sizes to fit the intensity and weight of the symptoms in the case.

Other types of windows include the following information which may be displayed to the user: Time window—Morning, Noon, Evening, Night, Dawn, Dusk, Periodicity, Hour format, a.m, p.m, twilight, forenoon, midnight. This information is preferably fed to various graphic displays, such as the circle of four elements and other systems of analysis. Other windows include a Weather window—Winter, Summer, Spring, Autumn, Weather, damp dry hot cold, storm, snow, rain; this information is preferably fed to Circle Generals; Menses window—Menses, Menstrual, Periods, Menopause, this information is related to Female genitalia; Sexual window—Erection, Masturbation, Coition; and Food window, which is preferably separated into four columns: desire column, aversions column, a column for food which aggravates, column for food which ameliorates. This four column arrangement may optionally also be used for other windows such as weather and time of day.

Optionally and preferably, all of the “boxes” (window sections and/or windows) may be viewed for a particular category simultaneously.

Preferably, data entry features a combination of structured fields and free text entry through a word processor. This word processor will be specially adapted to the homoeopathic process of case taking, including an autocomplete dictionary for completing words automatically after a few letters have been entered, and icons for marking various symptom attributes such as intensity.

According to another optional but preferred embodiment of the present invention, system 10 preferably features a library 24 for storing case histories of different patients, thereby allowing this information to be viewed when the patient comes for the follow up visit. One of the “views” of the case (the personal page view) preferably opens on recalling the case. This page preferably serves as the home page of the case, more preferably allowing direct access to all its other parts. Library 24 may optionally also feature general and/or materia medica information and texts.

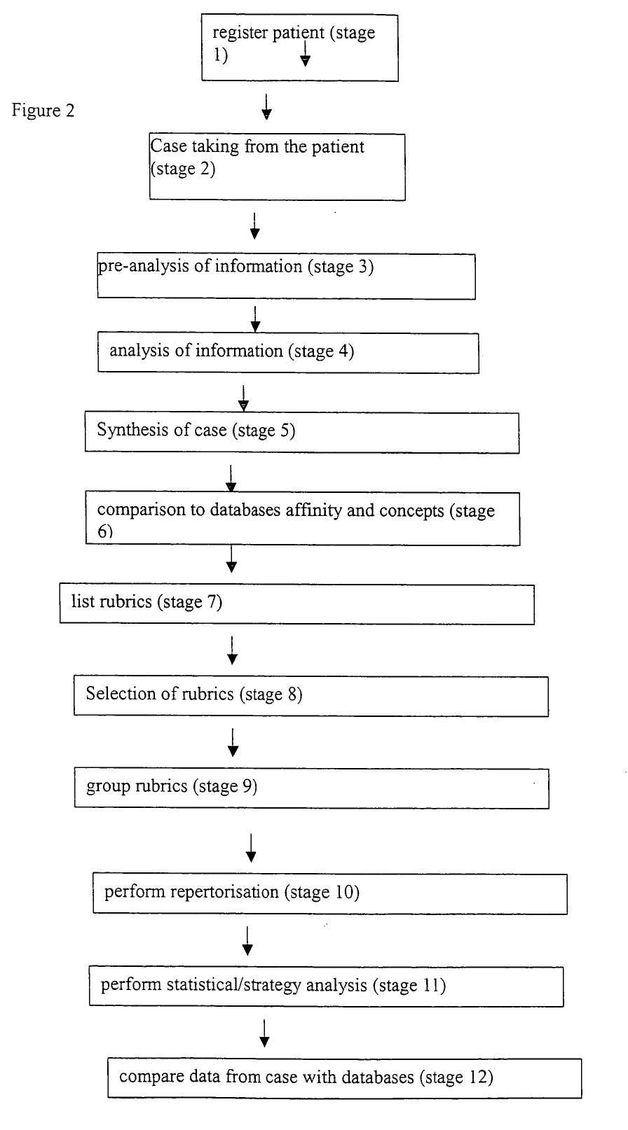

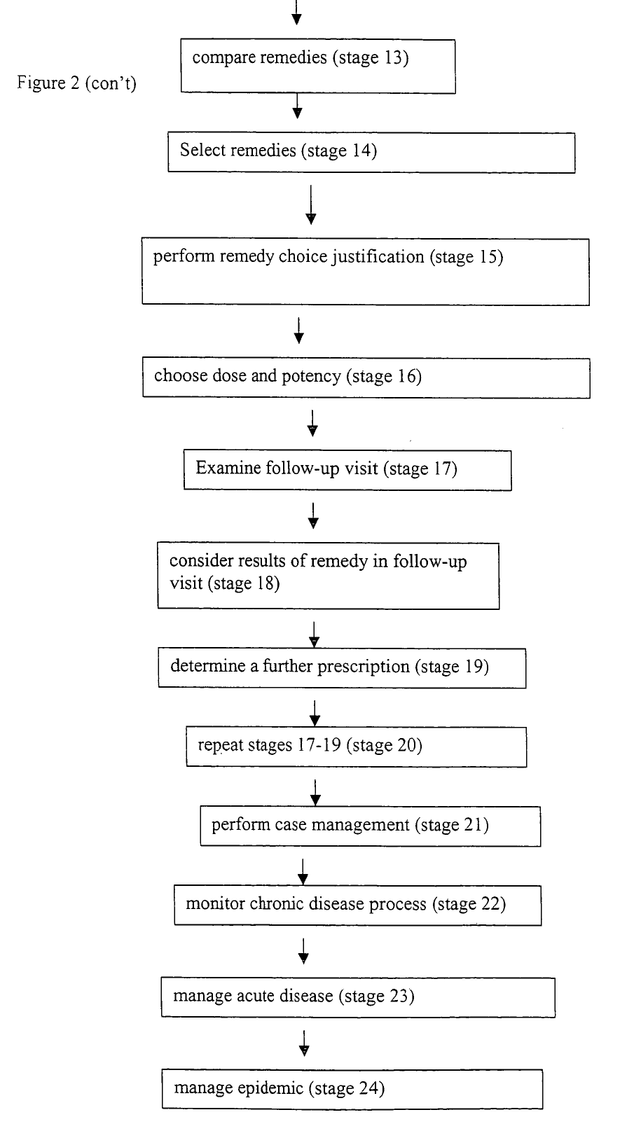

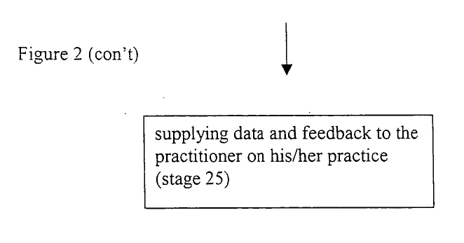

FIG. 2 shows an exemplary method according to the present invention. As shown, in stage 1 the patient is registered and personal details are obtained.

In stage 2, the practitioner performs the process of taking the case.

As the practitioner records and types in the patient's medical history, the information is preferably automatically, semi-automatically or manually sorted into homoeopathically relevant categories. The sifted and sorted information can optionally then be presented in a variety of views (windows) so as to enhance analysis of the data and its use in solving the case and also preferably for case management. For example, all pathological information may optionally be stored in one ‘window’. Related words and idioms are automatically grouped and stored in one window. All relevant life events may be displayed on a time line, since as described in greater detail below, the relative time at which particular symptoms occur is important. Logical connection and analogies are automatically detected or tagged, then grouped and displayed for easy reference. Diet and other lifestyle issues are sorted into their relevant windows. Other homoeopathically relevant information such as weather and temperature influences, times of day, appetite, sleep, emotional and intellectual symptoms and many others are sorted into categories. Body sections are sorted, such that all bladder symptoms preferably appear together. There are many other parameters by which data will be sorted, partly automatically and partly with some practitioner input.

According to optional preferred embodiments of the present invention, in order to assist the practitioner to enter the necessary information, preferably an electronic “tablet” and/or a PDA or any other type of handheld device or portable device, is provided as part of the system of the present invention, for enabling the practitioner to enter information with handwriting. Such tablets are well known the art, and may optionally be connected to a computer for example, in order for the handwritten notes of the practitioner to be automatically translated into data for analysis according to the present invention.

The entire case can then optionally be displayed, emailed or printed in a variety of modes. Databases of pathology, diagnosis and other relative data will be readily available in a computerized format.

The case taker preferably includes a module for inputting, storing and searching the patient's personal details. This module also preferably automatically calculates the patient's age and creating a time line. It also automatically generates a variety of more esoteric information based on the date of birth (astrology, Chinese astrology, numerology, i-ching, tarot, etc). These features are optionally and preferably displayed as part of the analysis view as described below.

According to preferred embodiments of the present invention, the practitioner may indicate preliminary considerations about particular words by changing the font and/or style of the text for example (bold for more emphasis for example), size to indicate intensity and so forth. The practitioner may optionally also select sentences according to these criteria. Preferably, the formatting of the text is performed automatically, once the practitioner has indicated the type of effect (intensity, emphasis etc) and its degree, for example by entering a symbol and/or by selecting a choice from a menu or other GUI (graphical user interface) gadget.

Preferably, one or more symbols may also be entered into the notes themselves, for example to indicate the nature of the expression on the patient's face when a word, group of words or sentence is spoken. Timing (rapid or slow speech, or hesitation) may also optionally be entered. These different types of annotations, optionally with other types of annotation, may be entered as well by the practitioner during case taking.

Optionally and preferably, the case taker is able to highlight words according to their frequency of use in the patient's native language (such as English for example) and/or in the general speech of the patient. More preferably, such highlighting is performed automatically.

Also optionally and preferably, a “box” or category may be provided for symptoms which have a connection in the speech of the patient, for example according to the manner in which the relevant words are spoken and/or according to accompanying facial expressions and/or gestures of the patient.

More preferably, for follow-up visits, a comparison may be performed between the word frequency of the patient's previous speech and the current speech of the patient. Also more preferably, such a comparison may optionally be performed as well for grammar.

Preferably, a dictionary is provided to assist the practitioner with word completion.

According to another optional embodiment of the present invention, the practitioner preferably is able to display a list of previously prepared questions, more preferably including questions previously prepared by the practitioner manually, most preferably according to the section or category of interest (for example the section or category currently being selected by the practitioner). Both the question and the patient's answer are preferably entered automatically to the note taking facility.

Once the medical data has been sorted (preferably at least partially automatically), stage 3 of the method of the present invention preferably allows further manipulation of the data so as to form an even more comprehensive analysis of the case. Stage 3 may also optionally be performed by the case taker module of the system according to the present invention.

Stage 3 preferably involves a pre-analysis stage, which enables the practitioner to focus their thoughts, and which preferably includes the determination of a plurality of parameters, more preferably no more than about 7 or 8 parameters that need to be considered. This provides a wide view of the case, and may optionally involve a text editing display, which may optionally guide the user, for example by alerting the user if too many parameters are selected. Optionally a list of parameters may be provided. Alternatively or additionally, a plurality of parameters may be automatically selected according to the method of the present invention. Other concepts which may optionally be considered include general health and prognosis, nature of any chronic illness, the presence of any pathologies, and vitality or energy of the patient.

These parameters enable the practitioner to have a large scale view of the patient before delving into the analysis and provide a buffer zone for assessing overview of the case, as most homoeopaths tend to jump straight into the search for a remedy or focus on minor issues, rather then assessing the case in its totality.

This assessment is also preferred for the case management stage (stage 21), and for the potency selector which shares some parameters (stage 16)

Scores (optionally from 1-10) are entered manually or automatically and are derived from the case taker.

The following parameters may optionally be used:

1. Health—overall assessment of health 1-10/10. 10 being totally healthy and 1 being very sick

2. Prognosis: assessment of future results and progress

a. without homoeopathy

b. with a good choice of remedy/remedies

3. Pathology; level of pathology eg slight pimple would rate 9/10, whereas heart attack and cancer would rate 2/10

4. Obstacles to cure: assessment of lifestyle condition that may impede cure, ie smoking, diet. Fewer obstacles=higher score

5. Affinities: as taken from affinity window in analysis

6. Miasms: according to homoeopathic philosophy, eg trends of chronic disease such as ‘syphilis’ or ‘Psora’

7. Totalities: is the case one whole entity or has it been broken down into a number of smaller totalities; for example if a new set of symptoms has arisen after a causative event such as head injury, they may be treated as a separate totality.

8. vitality. High vitality=10, low fluctuating or frantic vitality=lower score

In stage 4, an analysis stage is performed. The analysis process may optionally include clustering the symptoms. More preferably, the symptoms are clustered into weighted groups, according to the relative importance and/or likelihood of each cluster. This aspect of the present invention is particularly preferred since symptoms may optionally be featured in more than one cluster, although the suitability of such clusters may differ. Preferably, this stage is performed by an analysis module according to the method of the present invention.

The various windows (clusters, groups,) can be manipulated so as to enhance analysis and synthesis of the case. This will be aided by a variety of graphics.

Non-limiting examples of these windows include windows for mental, general and particular symptoms; sensations and modalities (the latter increase or decrease the perceived affect of symptoms); unusual or rare symptoms; general observations of the practitioner; all affinities; time of day/year or periodicities; weather, including temperature, light etc; menses and gynecological symptoms; sexual symptoms; food related information; remarks for case management, include extra details; obstacles to proper treatment; previous diagnoses received by the patient; idioms used by the patient; causations and other mental concepts of the patient; grammatical concepts; analysis and remarks of the practitioner; health or vitality levels; previous medical treatments received by the patient; personal and family history; lifeline and other timelines; main complaint of the patient; possible questions to ask, management comments, possible remedies, analysis comments, mental concepts, esoteric information and user defined windows.

It should be noted that some of the information may appear in more than one window.

Also optionally and preferably, the flow of information entry is maintained as the information is being copied to the previously described boxes, so that at this stage (or alternatively or additionally another stage), the practitioner may also view the flow of information input, in addition or in replacement for viewing the information by categories or boxes.

In stage 5, a synthesis process is preferably performed, in which the practitioner is prompted and aided in searching for a precise definition of the illness (Homoeopathic diagnosis or in homoeopathic terminology ‘what is to be cured’ (Organon of Medicine by Samuel Hahnemann 6^{th} edition).

Synthesis preferably also involves a synopsis of the patient's condition. As for the pre-analysis stage, the practitioner is preferably forced to analyze the condition in a specific manner; however, while the pre-analysis stage preferably involves a general consideration of the condition of the patient, the synthesis stage preferably features the use of affinities and concepts, as well as sensations and functions, chief complaint, circle of elements (the verb), kingdoms and the periodic tables, keywords, and other known methods of analogy and perception, which are specific homoeopathic concepts. The synthesis process may optionally involve recommending such affinities and concepts, and/or may assist the practitioner with selection from a list.

Other optional examples of processes which may be performed during synthesis include but are not limited to, analyses of the words of the patient. For example, optionally and preferably a word count may be performed with the words of the patient, more preferably including a grammatical analysis and/or language analysis. The grammatical analysis may optionally be used in order to help discover or uncover relationships between words in the mind of the patient. Language analysis may optionally be used in order to determine to which categories the words of the patient should be assigned. These types of analyses are also preferably used for one or more of the later optional statistical analyses which may be performed, for example in order to normalize or otherwise properly weight the statistical analysis.

In order to assist with the synthesis process, optionally and preferably the system according to the present invention also features a new database of meta-concepts and physical affinities, in addition to databases which feature individual symptoms.

In stage 6, in order to assist with the analysis, synthesis and remedy finding processes, optionally and preferably the system according to the present invention also features a new database of meta-concepts and physical affinities, optionally in addition to existing databases which feature individual symptoms. The concepts and affinities of the case will be automatically derived from the case and compared with these databases. For example, lists of remedies may optionally be compared with lists of symptoms in order to obtain a repertory of affinities and concepts. Some remedies are part of a generic family, which is preferably also displayed (for example, instead of a species of snake being relevant to a remedy, any type of snake may be relevant as part of the generic family of the remedy. For example the remedy Lachesis, which is a snake remedy, if applicable to the snake family as a whole, will be designated lach-x rather then just lach. This will cause all remedies in the snake family to be designated.

Homoeopathic affinity is a physiological location or system, eg, throat, ear, foot, digestion, metabolism, reproductive and so forth, as described in greater detail below. A homoeopathic concept is a generalized issue which characterizes the mental and emotional nature or pathology of the patient, ie religion, victim, forsaken, fearful as described in greater detail below. These meta concepts and affinities are arranged in a new database, each comprising two rubrics, one primary essential rubric and another secondary and more generalized rubric, which can be manipulated manually and automatically according to the weight and intensity of concepts and affinities in the case.

In stage 7 a full list of possible rubrics is selected from the existing rubric database, automatically and semi automatically, partly by designation and word recognition during the case taking process. In stage 8, assistance is provided in selecting the most efficient rubrics and combination. Rubrics are preferably selected according to the analysis provided during the synthesis stage, according to the clusters of the symptoms that are determined in that stage. As previously described, symptoms may potentially be associated with more than one rubric, yet not all such associations are likely to be correct.

In stage 9, the rubrics are preferably grouped. There is a science and art to choosing a grouping of rubrics. This process is preferably performed by a rubric selection module which provides guidance and feedback on the choice of rubrics according to the system of the present invention. The guidelines for grouping rubrics preferably comprise a plurality of rules, for example to prevent the user from attempting to group a rubric with 4 remedies with a rubric having many remedies. Preferably such rules are guided by the principle (determined by the present inventor) of “the right rubric is the rubric with the right remedy in it”. The process may also optionally be performed graphically.

Examples of suitable rules, formulated and developed as part of the present invention, include but are not limited to:
1. Be absolutely certain symptoms are true to case
2. Choose meaningful symptoms
3. Choose SRP symptoms
4. Minimum symptoms of maximum quality
5. Group sizes should be suitable (preferably approximately 10-100 remedies)
6. Choose reliable repertory rubrics
7. Do not overlap similar rubrics
8. If not sure combine rubrics rather then eliminate
9. Use proportional representation of schema
10. Use analogy
11. Do not rely on one repertorisation
12. Check all remedies covering 80% of rubrics, and those covering a lower percentage as more rubrics are added (if n is the number of rubrics, check all remedies representing 110%-10n % coverage)
13. Always check Materia Medica.
14. Read any remedy that is not known to the practitioner
15. Do not be excessively bound to a particular grouping.
16. The repertory is only an index, not a Materia medica

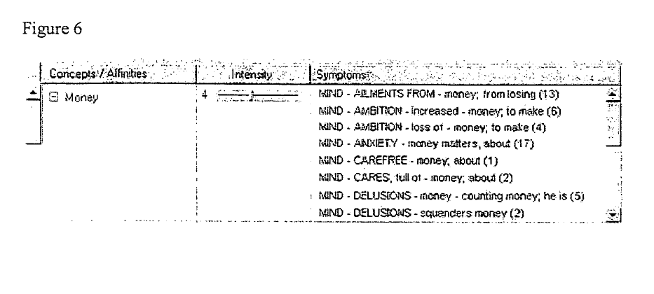

A possible example of how stages 7-9 may optionally function is given in the table below. This table is shown as a schematic diagram which could optionally be used for an exemplary screen shot, a non-limiting example of which is given in FIG. 6.

| | | | Button to analyse | | | | | | | | | |
|---|---|---|---|---|---|---|---|---|---|---|---|---|
| Concept | Symptoms | Intensity | 1 | 2 | 3 | 4 | 5 | 6 | 7 | 8 | 9 | 0 |
| +Jealousy | Symptom1 | -----6---- | X | | | X | | | | | | |
| | Symptom2 | --3------- | | X | | | | | | | | |
| | Symptom3 | --------8- | | | X | | | | | | | |
| | Symptom4 | -2-------- | X | | | | | | | | | |
| +Money | Symptom1 | -----6---- | X | | | X | | | | | | |
| | Symptom2 | --3------- | | X | | | | | | | | |
| | Symptom3 | --------8- | | | X | | | | | | | |
| | Symptom4 | --3------- | | X | | | | | | | | |
| | Symptom5 | --------8- | | | X | | | | | | | |
| | Symptom6 | -2-------- | X | | | | | | | | | |
| +Power | Symptom1 | -----6---- | X | | | X | | | | | | |
| | Symptom2 | --3------- | | X | | | | | | | | |
| | Symptom3 | --------8- | | | X | | | | | | | |
| | Symptom4 | --3------- | | X | | | | | | | | |
| | Symptom5 | --------8- | | | X | | | | | | | |
| | Symptom6 | -2-------- | X | | | | | | | | | |

[symbol]

----------scroll +/− consultations

In the example above words such as jealousy and money have been automatically or semi automatically exported from stages 2, 4, or 5, for example.

The present invention uses these words to search rubric databases and to list these in the list of symptoms which can then be chosen by ticking the appropriate boxes. Each list of symptoms is able to expand and contract, as well as the various consultations, thereby provided an easily accessible graphic tool to the practitioner.

Stage 10 preferably performs repertorisation, a process which is known in the art and for which software is available. Stage 11 may also optionally and preferably offer statistical and strategic analysis of the process, providing feedback and expert advice which can help the practitioner improve their choice of rubrics. These statistics are preferably performed according to rules of the repertorisation as detailed above For example, the statistical and graphical analysis will indicate the number of remedies in each rubric of a particular repertorisation, and the overplay between them. This will make it easy to detect any imbalance in repertorisation, for instance a rubric of 5 remedies with a rubric of 250 remedies, or two rubrics which do not overlap, ie two rubrics of 100 remedies with only 15% overlap which would be considered too low, or 85% overlap which would be too high. The graphic representation would also display the relative representation of the kingdoms of remedies, so as to easily see the percent of mineral, animal, vegetable and other groups which pass through each rubric and through the sum total. Other important information, for example miasmatic context of remedies, will be graphically and numerically indicated.

Examples of statistics formulated and developed as part of the present invention include but are not limited to: a display of the number of remedies in each rubric and the number of remedies passing through the group repertorisation, with graphical display of the overlap; a division of the number of rubrics per remedy into the number of words in its relevant proving, so as to indicate under and over repertorisation.

Examples of feedback include but are not limited to, determining whether a rubric is too large or too small; whether the rubrics are too closely related; and also whether a rubric is too new (in the sense that it has not been sufficiently tested by experience practitioners to be relied upon by all practitioners, or at least only to be considered with caution). Optionally, the determination of such characteristics as “too large” or “too small” for rubrics, and/or any of the previously described characteristics, may be performed according to one or more parameter(s) that are set by the practitioner.

In stage 12, data from the case analysis module is optionally (but not necessarily) automatically compared with existing databases and repertorisations to arrive at possible remedies. Such a selection may also optionally be made manually by the practitioner.

Optionally the results from the affinity and concept repertorisation may be displayed in conjunction with the conventional repertorisation (stage 10). In this concept and affinity repertorisation, preferably, in order to assist with this process, for each rubric a plurality of remedies is displayed which represent the essence of the rubric, as the most important group of remedies for the rubric. A larger group of remedies is preferably also displayed which represents all possible remedies for the rubric. During repertorisation, the practitioner may optionally slide between displays of these two groups.

Also optionally and preferably, in this stage keywords in the case taking notes of the practitioner are automatically or semi-automatically compared to words in the Materia Medica, using exsiting materia medica databases and search engines. Other optional but preferred comparisons include but are not limited to, comparing the number of words in the proving divided by number of rubrics in the repertory in order to achieve a balanced statistical analysis. For example, comparing the number of words in the provings to the number of rubrics preferably enables a statistical correction to be made for the size of remedies and their representation in the repertory.

In stage 13, preferably remedies resulting from the process are compared with the results of the analysis and synthesis stages of the case preferably from stages 4, 5, 6. Optionally and preferably, in this stage the relative percentage of miasms is compared between the case and the selected remedy, to determine whether they are relatively similar. For example, if the case has 80% psora and 20% psychosis, then preferably the selected remedy features at least similar percentages of psora and psychosis.

Stage 14 preferably assists with differential diagnosis of possible remedies.

Stage 15 preferably prompts the practitioner for remedy choice justification to enhance clarity and for further reference. This stage is particularly useful for tutoring students, although clearly the guidance described throughout the method of the present invention would also be useful for students and/or practitioners.

In the event that the remedy proves to be successful, notes from this justification and relevant notes from stage 5 can be exported to stage 28 (materia medica) so as to build a database of clinical information

Stage 16 facilitates the choice of dose and potency, by choosing potency based on laws formulated and developed as part of the present invention. These include a facility for assessing the health level of the patient according to various parameters by degrees from 1-10 (10 being best), for example as described with regard to pre-analysis stage 4. The scores may optionally be entered manually and/or automatically. These will be generated from the case taker (stage 2), the analysis module (stage 3) and preanalysis (stage 4), and include but are not limited to:
1. pathology—the level of pathology that the patient has as described above
2. modalities—a modality is a symptom modifier such as season, heat and cold, time of day, pressure, sleep etc; a well defined modality would score highly. eg headache worse every day at 4 p.m exactly would be a 9.5/10 whereas as poorly defined modality will be a low score, such as headache worse all day long would be a 3/10, a continuous headache would thus be a 1/10
3. Obstacles to cure: conditions which are part of the patient life and impede recovery, such as: smoking, drugs, nutrition, bad housing, radiation, overwork.
4. Mental/emotional states: the degree of freedom of the patient from mental emotional distress and pathology. For example a mild anxiety will score 8/10, whereas a severe schizophrenia will score 2/10.
5. Energy level: a high a consistent energy will score highly, eg. 9/10, whereas a low, fluctuating or frantic energy will have a low score eg 3/10
6. Freedom and creativity: a score will be given according to the patient's manifestation of their potential in life according to their own and their practitioner's estimation. For example a patient who has fulfilled all their dreams regarding work, relationship hobbies etc will score a high 9/10, whereas a patient with an antagonistic relationship and occupation who is not fulfilling their potential will score a low 2/10. This is in accordance with paragraph 9 of the Organon which states that a person's health will be fulfilling ‘the higher purpose of our existence’.
7. Sensitivity: a patient who is oversensitive to environmental factors and remedies will score low, whereas a patient who is totally insensitive to remedies and environment will also score low. A high score represents a well balanced and moderate sensitivity.
8. Practitioner experience: the more years of experience the practitioner has, the higher their score.
9. Remedy certainty: the higher the confidence in the remedy choice, the higher the score

These parameters can be added to and edited by the practitioner. The average and weighted score of the parameters will be compared to a sliding scale of potency so as to suggest a most suitable potency and repetition. The potency scale as compared to the average score will be able to slide up and down according to practitioner preference. ie one practitioner may equate a high score of 8 with a 10M (very high) potency, whereas another may prefer a 30C (medium potency) as their highest potency. These numbers refer to well known concepts for homoeopathic potency, such that the suffixes “M” and “C” refer to relative potencies.

The potency selector will also preferably and optionally advise a preferable regime of repetition of the dose.

The potency selector is preferably capable of intelligent learning: the case results will be fed back to the selector so that it may adjust its decision according to the result of multiple selections.

According to a preferred embodiment, a series of questions will be asked, the homoeopath will reply with a numeric value to each question. This will allow to calculate the mean value in order to highlight the corresponding area on the potency scale.

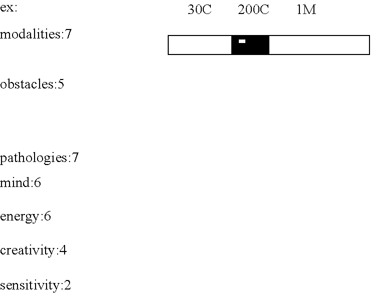

The scale can be customized.

In stage 17, follow-up visits to the practitioner by the patient are preferably examined in a specifically modified version of stage 2, the case taker. For example, optionally and preferably notes from the first and/or at least one previous consultation are provided for the practitioner to read. The practitioner more preferably is able to input comments as to changes observed in the patient within these notes; these comments are preferably then automatically transferred to the correct “boxes” as previously described, optionally also including a follow-up “box”. These changes may optionally and preferably include the percentage of improvement or deterioration, which can subsequently be used to track changes in symptoms throughout further follow-up consultations. These changes may also optionally and preferably be used for a total assessment of overall progress since the first consultation, and/or as a tool for selecting subsequent prescriptions.

In stage 18, once the patient returns, a comparison of the original case with results of the remedy is preferably performed, expressed by percentage of improvement or deterioration and optionally represented by graphs. Optionally and preferably, the percentage of improvement is characterized according to the criteria of rapid, gentle and permanent as determined in relation to paragraph two of the Organon. This stage then preferably facilitates the stage of choosing a new remedy.

Stage 19 will assist and provide expert advice in the selection of the second and subsequent prescriptions as well as by adjusting the rubrics selected in stages 8-10 according to the percentage or degree of improvement and deterioration, thus modifying the possible remedies to the patient's present condition. This stage preferably also considers and weighs new, old, cured and altered symptoms. The output is also optionally displayed graphically. Optionally and preferably this stage uses the relationship of remedies database. Guidelines from classical and contemporary homoeopaths are preferably displayed appropriately.

Stage 20 preferably involves a repeat of stages 17-19 for the purpose of all subsequent consultations.

Stage 21 preferably includes case management, which more preferably allows comments and hints on long-term case management and tracks the progress of the patient in comparison to conclusions derived in stages 3-5 and 17-20. This long term progress is preferably displayed in a variety of modes, such as by percentage of change and/or graphically, and also optionally by using displays such as a chess board in an analogy developed in conjunction with this invention.

Stage 22 involves monitoring of a chronic (mismatic) disease process, in order to determine remedies which are associated with chronic disease, and providing further guidance for the treatment of chronic disease, which would be related to all of the ramifications of chronic disease (miasms, acute, epidemics, stronger dissimilars and obstacles). A non-limiting example of a graphical representation of improvement or deterioration of a patient is a chessboard, for representing progress in the case to the practitioner through the GUI (graphical user interface) of the present invention.

Stage 23 enhances the treatment of acute disease, for example as described above. These stages preferably use relevant repertory techniques, databases, rules and guidelines, including those developed in conjunction with this invention. For instance complimentary remedies to those previously prescribed and repertorised are preferably automatically displayed, and opposite modalities appropriate to acute disease are preferably highlighted and weighted. Remedies more appropriate to acute conditions will be given extra weight.

Stage 24 (the epidemic stage) preferably similarly uses appropriate guidelines and techniques, including multiple files and clipboards to represent the large amount of patients suffering such a disease. Preferably and optionally, the software, utilizing stages 1-15 will automatically or semi automatically compile and analyze the epidemic information from multiple patients by body parts, affinities and concepts, creating a larger totality in order to identify the ‘genus epidemicus’ (group of remedies suitable to this particular epidemic), as described by Hahnemann in the Organon of medicine paragraphs 73, 100, 101, 102. This genus epidemicus will be selected from a weighted repertorisation, so as to avoid arriving only at large remedies. Consequently the software will optionally and preferably help to select individual remedies from the genus epidemicus group, also by recalling each particular case that has been repertorised and stored, and comparing the individual symptoms to the genus epidemicus remedies. The software will optionally and preferably be capable of automatically updating and adjusting the genus epidemicus with each extra case seen, utilizing as well the data from cured cases. This data will be shared by users over the internet so as to automatically and semi automatically enlarge the database of information relevant to the epidemic. In performing these functions the software will refer to existing databases of remedies, repertories, affinities, concepts and relationship of remedies, as well as displaying appropriate advice from classical and contemporary sources.

Stage 25 preferably supplies data and feedback to the practitioner on his/her practice during the session and also for case management, and/or for any other aspects of the homoeopath's practice. The method at this stage preferably enables automatic monitoring of results, trends and patterns in terms of the patient's details such as age and professions, remedies, potencies, remedy results, number of rubric taken, remedy chosen to remedies suggested, potency chosen to potency suggested, number of consultations etc. Every stage of the module preferably provides feedback and is preferably and optionally displayed in numerical, percentage, verbal and graphical manner so as to facilitate a continual overall assessment of the practice.

Stage 26 preferably enables the practitioner to receive information about the recommendations of other practitioners for patients having the same or similar symptoms. For example, the present invention may optionally connect to a database, for example through a network such as the Internet. Optionally, the practitioner is only allowed to access the database if the practitioner also supplies information to the database, preferably coded to maintain the privacy of patients and the confidentiality of the practitioner/patient relationship. Therefore, in this stage the practitioner may optionally perform data mining for example, in order to learn more about how particular remedies are handled, for example in terms of dose and/or potency for example. The database may also optionally include a knowledge base for assisting the practitioner, based on an analysis of previously received case intake data. Preferably, a subscription would be required for the practitioner to access this database. Preferably practitioners connected to this network are be able to feedback and make suggestions to the program, as well as to specific observation and guidelines they have developed, which may optionally be incorporated into the program.

Stages 27-29 are not all necessarily performed, nor are they necessarily performed sequentially (with each or with other stages of the method as previously described); they represent different options for augmenting the method of the present invention.

Stage 27 is optionally performed to assist the practitioner if all attempts at selecting a treatment and/or previous treatments fail. In this stage, alternative pathways are suggested if previous suggestions fail. For example, this stage optionally and preferably features reviewing the selections and choices made by the practitioner, in order to suggest an additional question to ask, and/or a different way to analyze the information and/or a different pathway to follow to select a rubric and/or remedy. For example, the practitioner may optionally be provided with a flowchart of different possible pathways and methodologies to consider and preferably their potential consequences in terms of treatment. An option to post the case on a user group Internet or intranet site is optionally and preferably available.

Stage 28 provides an option for adding personal materia medica information collected and confirmed from one or more of stages 3, 5, 12, 13, 14, 15, 16, 17, 18, 19, 20, 22, 23, 24 or 25. The information is stored, sorted analysed and synthesized, optionally using software developed for stage 2, 3, and 5. Optionally and preferably tools are provided for easy conversion to rubric form to be exported to personal and collective repertories. All this information is exportable and optionally available for the user group described above.

Stage 29 preferably involves adding data from a new proving. As previously described, each proving preferably features gathering data from the effect of a substance on a plurality of healthy individuals, and then analyzing the effect(s) to determine the potential use(s) of the substance for treating patients. Stage 29 may optionally be supplied through an additional module, which would enable intake of all of the data on the effect(s) of the substance and analysis of the data. This module may optionally be a “stand-alone” module, as stage 29 is optionally performed separately, without any connection to the rest of the previously described method of the present invention.

In order to assist the practitioner to add such data for a new providing, optionally the previously described text editor for case intake is used (stages 2-5), for example in order to allow the practitioner to add the effects of the new proving through “boxes” as previously described, in order to be able to sort these effects into different categories. The case taker module can therefore optionally be used to sort through this large mass of data in order to organize a proving. Alternatively, a separate proving analyzer/organizer may optionally be provided (not shown). The data which is then analyzed and sorted is optionally available for export to the user group through the network (Internet or intranet).

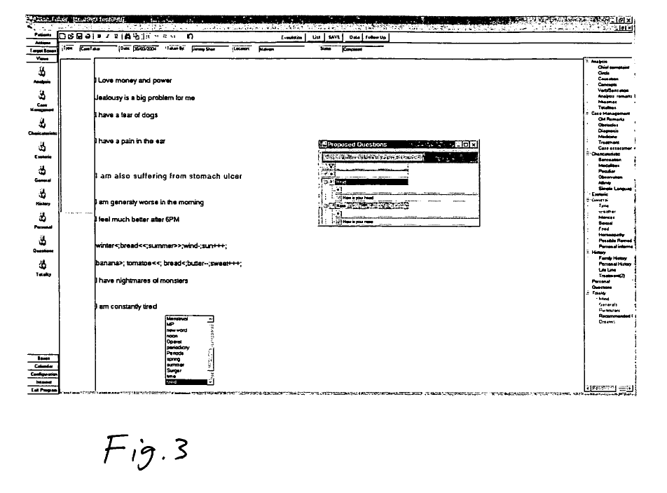

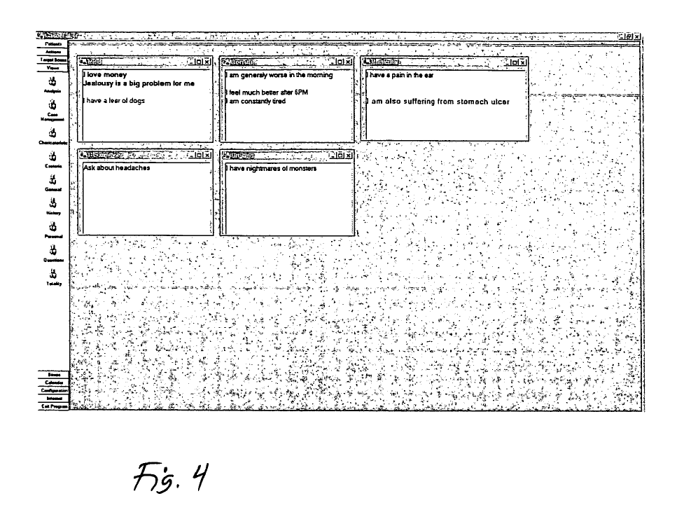

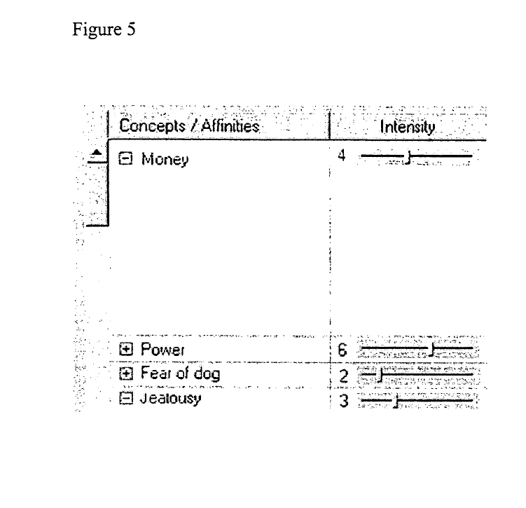

According to preferred embodiments of the present invention, the practitioner is able to enter data through a GUI (graphical user interface) as described above. FIG. 3 shows an exemplary, non-limiting window for the GUI for the case taker module. The practitioner is able to enter key words or concepts, such as money, power, fear of dog and jealousy. FIG. 4 shows automatic and/or semi-automatic distribution of symptoms into boxes (windows) according to context. These symptoms are then preferably automatically or semi-automatically exported to the GUI as shown in FIG. 5.

FIG. 5 shows an exemplary window in such a GUI, in which the practitioner is able to select concepts and affinities, such as money, power, fear of dog and jealousy on the left hand side, and the relative intensity of such concepts and affinities on the right hand side through a slider bar selection “gadget” or device. FIG. 6 shows the ability of the practitioner to further select and/or enter symptoms according to such concepts and affinities, as shown with regard to the example of money.

According to another optional embodiment of the present invention, there is provided a method for organizing the free flow of information for any type of consultation in which a case is considered according to communication between two humans, for example for professional work as previously described. The present invention may also optionally be implemented for any type of interviews or examinations. According to another example, the method is implemented for law. Briefly, the legal practitioner meets with a customer, and first receives basic information about the customer. Next, the practitioner receives information about the customer's activities; for example, for a criminal case, the practitioner would need to receive information about all activities that could be related to the crime, but also preferably receives general information about all other activities of the customer. Next, first a general overview is considered, preferably at least semi-automatically. Next, the details are preferably arranged for a comprehensive picture of all legal issues facing or of concern to the customer. A synthesis is then preferably performed, preferably forcing the practitioner to focus upon the specific legal situation. Possible courses of action are provided, after which preferably one course is selected and is compared to the previously obtained information. The method may also optionally involve varying interpretations of the information, for example to automatically provide information about relevant case law and statutory law. Other relevant information, such as the behavior of judges in the local area, may also optionally be provided.

While the invention has been described with respect to a limited number of embodiments, it will be appreciated that many variations, modifications and other applications of the invention may be made.

1. A method for organizing the free flow of information during a consultation on a case, comprising:
organizing the free flow of information obtained through the communication according to a plurality of levels, including at least one general level and one specific level;
recommending at least one solution;
checking said at least one solution against the free flow of information and said organized information; and
optionally indicating at least one feature of the case that is not suitable for said at least one solution.

2. The method of claim 1, wherein the method is suitable for medical practice (in which the case is a patient), legal practice (in which the case could be a legal or court case, a patent application, a trademark application, a legal opinion and so forth), professional consulting such as for architecture (in which the case could be a building to be built or modified), psychological counseling and education, or any kind of free flowing information that is to be sorted.

3. The method of claim 1, further comprising:
assisting with follow-up and long term management of a case.

4. A method for assisting a homoeopathic practitioner with a patient, comprising:
performing case taking with the patient to receive information about the symptoms;
organizing the symptoms according to a pre-analysis;
specifying an essence of a condition of the patient according to said organized symptoms; and
displaying a plurality of rubrics according to said essence of said condition.

5. The method of claim 4, further comprising:
grouping said rubrics according to said essence of said condition and the symptoms;
choosing at least one remedy; and
comparing said at least one remedy to said essence of said condition and the symptoms.

6. The method of claim 5, further comprising:
justifying a selection of said at least one remedy according to said essence of said condition and the symptoms.

7. The method of claim 6, further comprising:
diagnosing an acute condition, a chronic condition and an epidemic; and
examining a selected remedy to determine suitability for acute and chronic conditions and/or epidemics.

8. The method of claim 7, further comprising:
analyzing further information provided by the patient in a follow-up visit.

9. The method of claim 8, further comprising:
selecting a new remedy according to said further information; and
comparing said new remedy to said remedy being previously selected.

10. The method of claim 5, further comprising:
selecting a potency of said remedy according to at least one rule.

11. The method of claim 5, wherein said comparing is performed according to analysis of information from a plurality of different practitioners.

12. The method of claim 1, further comprising:
providing feedback according to said analysis.

13. The method of claim 12, wherein said feedback includes teaching information for a student practitioner.

14. The method of claim 4, further comprising:
providing feedback to the practitioner about said essence of said condition.

15. The method of claim 14, wherein said feedback is based upon statistical analysis of information from a plurality of previous consultations with different patients.

16. The method of claim 15, wherein said previous consultations are from different practitioners.

17. The method of claim 4, further comprising:
providing a dictionary for automatic word completion of at least one word at least during said case taking.

18. The method of claim 4, wherein said case taking further comprises using at least one graphical tool for manipulating at least one word.

19. The method of claim 4, wherein said organizing the symptoms further comprises tagging symptoms for correlation between said organizing and at least one stage of analyzing, selecting a remedy or selecting a potency.
